(19) United States
(12) Patent Application Publication
(10) Pub. No.: US 2006/0188548 A1
(43) Pub. Date: Aug. 24, 2006

(54) SATIETY PROMOTING BEVERAGE AND USE IN A DIET TO MODERATE FOOD CONSUMPTION

(76) Inventors: Peter H. Mattson, Hillsborough, CA (US); Connie J. Gregson, Langhorne, PA (US); Gregory T. Horn, Lighthouse Point, FL (US); Michelle J. Regan, Castro Valley, CA (US)

Correspondence Address: THADDIUS J. CARVIS, 102 NORTH KING STREET, LEESBURG, VA, 20176, US

(21) Appl. No.: 11/275,130

(22) Filed: Dec. 13, 2005

Related U.S. Application Data
(60) Provisional application No. 60/635,918, filed on Dec. 14, 2004.

(51) Int. Cl. A61K 36/82; A61K 47/00; A61K 36/18

(52) U.S. Cl. 424/439; 424/729; 424/777

(57) ABSTRACT

A satiety promoting beverage and a weight control regimen using it are described comprising: fiber, protein, sweetener and preferably calcium and a catechin, such as purified epigallocatechin gallate derived from green tea. The ingredients are employed in amounts sufficient that a single serving provides a feeling of satiety, both initially and after a period of at least one hour. In a preferred form, a serving of the beverage will comprise fiber in an amount sufficient to supply from 10 to 30% of the DV for fiber; milk protein in an amount sufficient to supply from 4 to 10 grams protein; calcium in an amount sufficient to supply from 10 to 40% of the DV for calcium; a low calorie sweetener and, preferably, the catechin in an amount sufficient to supply from 2 to 4 cup equivalents. The ingredients are homogenized and packaged to provide a smooth textured beverage with a creamy mouthfeel and from about 50 to about 150 calories per serving of from about 4 to 12 ounces. A Fullness Index of greater than 4, and preferably 5 is evidence of both very high volume to calorie and (fiber plus protein) to calorie ratios.

### RELATED APPLICATIONS

This application claims priority to prior U.S. Patent Application No. 60/635,918 filed Dec. 14, 2004, which is incorporated herein by reference in its entirety.

### BACKGROUND OF THE INVENTION

The invention relates to a satiety promoting beverage and its use in a diet to moderate food consumption; and in particular to a beverage composition including a highly-effective combination of macro- and micronutrients that can induce satiety in human subjects.

Food is essential to life. It adds to the richness of the human experience. Yet, people tend to eat too much, and limiting consumption can affect the quality and length of the life. There are known factors that help extend life, and there are at least as many that can shorten it, one of the negative factors being over consumption of foods. See, generally, The Surgeon General's Report on Nutrition and Health. Washington, D.C.: US Dept of Health and Human Services; 1988 and DHHS(PHS) publication No. 88-50210. The Surgeon General's Report states, “For two out of three adult Americans who do not smoke and do not drink excessively, one personal choice seems to influence long-term health prospects more than any other: what we eat.” (p 1). The report goes on to point out that, while under nutrition remains a problem in many parts of the world including for some Americans, the more likely problem for Americans is the tendency to eat too much. The type and amount of foods are implicated in weight control and general health. The situation has not improved since the report was issued in 1988.

People know that they should try to eat less, but many have difficulty given the typical western lifestyle in which people can eat as much as they want. Healthy, sustainable weight management isn't just about how many calories you eat—it's also about eating food that's great tasting and satiating, so a person doesn't tend to overeat.

Over a recent passage of twenty years, obesity increased to epidemic levels. According to the National Institute of Diabetes and Digestive and Kidney Diseases (NIDDK), about 64% of adults (130 million adults over the age of 20) are overweight and 30% (61 million adults) are obese. Under a third of all U.S. adults have a healthy weight. Amongst children, the rate of increase in obesity is especially alarming. Between 1980 and 2000, the percentage of overweight children more than doubled to 15%.

Millions of Americans are dieting and will continue to invest their money to lose weight. Unfortunately, much of their investment produces little gain toward a lower weight and better health. Weight control and diet formulations have been one area where weight conscious individuals have looked, however, these formulations are often not perceived as healthy. In fact, in April 2004 the U.S. FDA banned one such group of products, those containing ephedrine alkaloids, citing a link to increased risk in heart attacks and strokes. This action left many weight conscious consumers without their favorite diet medication and further fueled a controversy surrounding dietary supplements. Many are justifiably wary of unfamiliar, new dietary supplements that promise results that seem unreasonably good.

The cyclic emergence of fad diets has preyed upon the millions of Americans who are trying to lose weight or prevent weight gain. What is needed, however, is an approach that provides a reliable moderator to the person wanting to truly enjoy food as one of the joys of life. Diet supplements, pills or regimens that are intended as stand-alone solutions to weight loss and weight management are not available with a simple self regulatory feature. In all cases weight control consistent with health should be part of an integrated weight loss and weight management plan which includes healthy eating and regular exercise. What is needed is a food supplement, which, instead of making unrealistic and false weight loss claims, will help a weight conscious individual with appetite control.

Understanding food consumption in terms of digestion and satiety is not a matter simply summarized or outlined. The development of effective diet foods and regimens has lagged behind breakthrough understandings based upon a vast amount of research. There is a burning desire for the achievement of some means for people to be able to enjoy food for what it adds to life while sustaining it, while not overeating on a regular basis.

It is known that the gastrointestinal presence of ingested nutrients initiates a range of physiological responses that serve to facilitate the overall digestive process. During a meal, ingested nutrients accumulate in the stomach, with a significant portion passing on to the small intestine. Thus, peptides and transmitters are released, and various neural elements are activated that coordinate gastrointestinal secretion and motility and can eventually lead to meal termination or satiety. See Cees de Graaf, et al., Am J Clin Nutr 2004; 79:946-61, wherein the authors note that the term “satiation” means the state of being comfortably full and that “fullness” refers to the sensation of fullness in the stomach. They also note that “hunger” is typically considered the opposite of “satiety”. Herein we take the terms satiety and fullness to be the same. Among the range of gastrointestinal peptides released by ingested nutrients is the brain/gut peptide CCK (Cholecystokinin). Moran T H and Kinzig K P (Gastrointestinal satiety signals II. Cholecystokinin, Am J Physiol Gastrointest Liver Physiol. 2004 February;286(2):G183-8.) explain that CCK can play a variety of roles in coordinating gastrointestinal activity and has been demonstrated to be an important mediator for the control of meal size. And, Moran T H, (Cholecystokinin and satiety: current perspectives, Nutrition, 2000 October;16(10):858-65.) concludes that a physiologic role for endogenous CCK in the control of meal size has been demonstrated and sites and mechanisms of action for CCK in food intake have been investigated. They also report that recent work has uncovered roles for the CCK satiety pathway in the mediation of the feeding modulatory actions of estradiol, insulin, and leptin.

Consumption of foods in general will cause a person to feel full or satiated as eating progresses. Once a person is satiated, the inclination moves from eating to rest from eating. Proteins, minerals, fiber, carbohydrates, and fats all have effects on this phenomenon. Satiety can be measured both subjectively by noting what a subject feels like and analytically by looking at certain biochemical markers. One such marker often studied is CCK (cholecystokinin) and another is GLP-1 (glucagon-like peptide-1). Studies have shown that protein, a satiating macronutrient, may differ in its effects on appetite depending on the protein source and variation in digestion and absorption. Hall W L, Millward D J, Long S J, Morgan L M. (Casein and whey exert different effects on plasma amino acid profiles, gastrointestinal hormone secretion and appetite, Br J Nutr. 2003 February;89(2):239-48) investigated the effects of two milk protein types, casein and whey, on food intake and subjective ratings of hunger and fullness, and on postprandial metabolite and gastrointestinal hormone responses. They conducted two studies that implicate post-absorptive increases in plasma amino acids together with both CCK and GLP-1 as potential mediators of the increased satiety response to whey and emphasize the importance of considering the impact of protein type on the appetite response to a mixed meal.

Likewise, fiber is a satiating food, if not macronutrient. Cholecystokinin is associated with satiety. Fat stimulates cholecystokinin release, and fiber appears to prolong cholecystokinin elevation during the alimentary period. See, for example, Burton-Freeman B, Davis P A, Schneeman B O (Plasma cholecystokinin is associated with subjective measures of satiety in women, Am J Clin Nutr. 2002 September;76(3):659-67) They found that in women, the feeling of satiety caused by cholecystokinin release is enhanced by increasing either the fiber or fat content of a low-fat, low-fiber meal. In the men, the increase in cholecystokinin concentration did not differ between meals, but the two low-fat meals elicited a greater feeling of satiety than did the high-fat meal. In another study, by Bourdon I, Olson B, Backus R, Richter B D, Davis P A, Schneeman B O (Beans, as a source of dietary fiber, increase cholecystokinin and apolipoprotein b48 response to test meals in men, J Nutr. 2001 May;131(5):1485-90) it was concluded that adding beans to a meal to increase fiber content prolongs the postprandial presence of intestinally derived lipoproteins and augments the CCK response to the meal.

Various herbal remedies have been suggested in micro amounts as providing a wealth of health benefits, as have some mineral supplements. Green tea polyphenols, especially the catechin, (−)-epigallocatechin gallate (herein, simply “epigallocatechin gallate” or EGCG), have been proposed as a cancer chemopreventative based on a variety of laboratory studies. To make a clear assessment of the possible physiological effects of green tea consumption, Kao Y H, Hiipakka R A, Liao S. (Modulation of endocrine systems and food intake by green tea epigallocatechin gallate, Endocrinology. 2000 March;141(3):980-7) injected pure green tea catechins into rats and studied their acute effects on endocrine systems. However, Kao et al. found that EGCG, but not related catechins, significantly reduced food intake. In other studies on green tea extract, Dulloo A G, Duret C, Rohrer D, Girardier L, Mensi N, Fathi M, Chantre P, Vandermander J. (Efficacy of a green tea extract rich in catechin polyphenols and caffeine in increasing 24-h energy expenditure and fat oxidation in humans, Am J Clin Nutr. 1999 December; 70(6): 1040-50) found that green tea has thermogenic properties and promotes fat oxidation beyond that explained by its caffeine content per se. The green tea extract may play a role in the control of body composition via sympathetic activation of thermogenesis, fat oxidation, or both.

Calcium is essential for the formation and maintenance of bones and teeth and has been associated with a variety of other functions in the body. It has, for example, been associated with satiety, preferentially in the form of milk. See, for example, Zemel M B, et al. (Dietary calcium and dairy products accelerate weight and fat loss during energy restriction in obese adults. Obesity Research. 2004; 12(4): 582-590) who reported that obese people who consumed three to four daily servings of milk, yogurt or cheese while on a balanced, reduced calorie diet, lost significantly more weight and fat than those who consumed similar amounts of calcium through supplements, or who consumed one or fewer servings of milk, yogurt or cheese per day. People on the high dairy (1200-1300 mg calcium) diet lost 70% more body weight and 64% more body fat than those on the low calcium diet. See also, Loos R, et al. (Calcium intake and body composition in the HERITAGE Family Study. Obesity Research. 2003; 11(S): 597-P) where data from over 800 adults enrolled in the Study was evaluated and found that high daily calcium intake (including from dairy foods) is associated with lower weight. In like manner, Teegarden D, et al. (Symposium: Dairy product components and weight regulation. Journal of Nutrition. 2003; 133: 243S-256S.) concluded from a research review that nutrients found in dairy, including calcium, may contribute to the reduction of body weight, body fat and insulin resistance syndrome. See also, Schrager, The Journal of the American Board of Family Practice 18:205-210 (2005).

The art has endeavored to develop weight control foods for increasing the sense of satiety of a subject wishing to diet, but the formulations have all left something to be desired in terms of desirability, conditions of consumption or formulation of effective ingredients.

The science of nutrition and the availability of biomarkers to assess the fate of food processed by the human organism helps understand the overall scope of the problem, which is all too practical and real to the weight conscious individual. The art is in need of new products which can offer a greater chance that a reliable regimen can be established and maintained by weight conscious individuals. A product that is lacking in the art is one that provides satiety promoting properties sufficient to moderate food consumption and yet is so good tasting that people will look forward to a snack based on it. Also needed is a food supplement, which, instead of making unrealistic weight loss claims, will help a weight conscious individual with appetite control. The art is in need of a weight control regimen which includes a satiety promoting product that is so satisfying on its own that weight control will naturally follow.

### BRIEF DESCRIPTION OF THE INVENTION

It is an object of the invention to provide a delicious, creamy beverage, which is low in calories and provides a useful degree of satiety upon consumption.

It is an object of the invention to provide an all natural beverage, which offers satiety promoting properties sufficient to moderate food consumption and yet is so good tasting that people will look forward to a snack based on it.

It is another object of the invention to provide a beverage providing less than or equal to about 150 calories per serving, e.g., less than about 100, and preferably less than or equal to about 80 to about 90 calories per serving, which provides a useful degree of satiety upon consumption.

It is another object of the invention to provide a beverage providing a low caloric density, which can be useful in a weight control regimen by providing a significant degree of satiety that will naturally discourage over-consumption.

It is another object of the invention to provide a beverage having a low caloric content, which instead of making unrealistic and false weight loss claims will help a weight conscious individual with appetite control.

It is another object of the invention to provide a beverage having a low caloric content, which provides dietary advantages such as the inclusion of significant levels of calcium, fiber and, preferably, antioxidants, such as from green tea extract, so as to encourage consumption to thereby help avoid undue hunger between meals while providing other health benefits.

It is another object of the invention to provide a beverage having a low caloric content, which contains micro and macronutrients in a highly palatable form that can help a weight conscious individual avoid undue hunger between meals.

These and other objectives are accomplished by the invention which provides a satiety promoting beverage, a method of using the beverage in a regimen to moderate food consumption, and to specific beverage compositions including effective combinations of macro- and micronutrients that can induce satiety in human subjects.

In one aspect, the invention provides a satiety promoting beverage, comprising: fiber, protein, calcium and sweetener, the ingredients being employed in amounts sufficient that a single serving will provide a feeling of fullness and satiety to the person consuming it, both initially and after a period of at least one hour.

In one preferred embodiment of the beverage, the invention provides a satiety promoting beverage, comprising: fiber, protein, calcium and sweetener, wherein each serving of from about 4 to about 12 fluid ounces contains more than about 4 grams of protein, more than about 4 grams of fiber, with a combined amount of protein and fiber of from about 10 to about 16 grams, and provides less than about 100 calories and has a caloric density of from about 4 to about 10 calories per fluid ounce. As a variation on this approach, the calorie content can be increased where it is desired to use a sugar as the sweetener.

From another perspective, the invention provides a satiety promoting beverage, comprising: fiber, protein and sweetener, the ingredients being employed in amounts sufficient to provide a Fullness Index of at least about 4. The following expression describes the Fullness Index (FI) using notation from an Excel spreadsheet for calculating its values.
FI=(41.7/(Aˆ0.7))+(0.05*B)+(6.17*(EXP(−4))*(Cˆ3)−(7.25*(EXP(−6))*D)+0.617)

where for each 100 g of beverage,
A is calories
B is grams protein
C is grams dietary fiber, and
D is grams fat.

In another aspect, the invention provides a regimen for weight control, which comprises: prior to a meal, consuming one satiety promoting beverage comprising fiber, protein, calcium and sweetener, wherein each serving of from about 4 to about 12 fluid ounces contains more than about 4 grams of protein and more than about 4 grams of fiber, with a combined amount of protein and fiber of from about 10 to about 16 grams, and provides less than about 100 calories and has a caloric density of from about 4 to about 10 calories per fluid ounce.

Many preferred features of the invention will be described below.

### DETAILED DESCRIPTION OF THE INVENTION

The invention enables maintenance of a non restrictive diet by aiding a weight conscious consumer in easily limiting calories. And, it does so while supplying macronutrients without the use of unproven drugs or the elimination of entire meals. When the beverage is properly prepared in accord with the following description of exemplary formulations and procedures, the weight conscious consumer will absolutely want to have the delicious snack break and will, therefore, find it easy to maintain a dietary regimen that will help meet an important goal of sensible eating at main meals.

The invention provides a satiety promoting beverage, which is delicious and effective for aiding in weight control by enabling people to control the amount they eat while allowing them to eat with their friends and family as they would normally. From the standpoint of formulation, the beverage will comprise, in addition to water or other aqueous base: fiber, protein, especially from milk, calcium, sweetener and, preferably, antioxidant compounds such as catechins, especially purified epigallocatechin gallate derived from green tea. These ingredients are employed in amounts sufficient for a single serving to provide a feeling of fullness to the average consumer. The satiety will be felt both initially and after a period of time, e.g., at least about one hour, and preferably for about two hours.

A primary aim of the invention is to aid the weight conscious individual utilizing it to make weight control more simple and more pleasant. By providing an immediate satiety response to consumption, the person can drink it directly before eating and have a benefit of eating less than normal. By providing a long lasting response, timing of consumption should not be a separate cause of stress. Preferably, the amounts of the various ingredients will be effective to result in the person consuming the beverage taking in less food in terms of weight and/or calories. The amounts of the individual ingredients will preferably together provide a measurable feeling of satiety and an increase for the combination in CCK serum levels. Because satiety is considered a pleasant sensation of fullness, the terms “satiety” and “fullness” are used as synonymous in meaning herein.

A Fullness Index has been adopted to help objectively compare various formulations to predict their effect on satiety. The following expression describes the Fullness Index (FI) using notation from an Excel spreadsheet for calculating its values.
FI=(41.7/(Aˆ0.7))+(0.05*B)+(6.17*(EXP(−4))*(Cˆ3)−(7.25*(EXP(−6))*D)+0.617)

where for each 100 g of beverage,
A is calories
B is grams protein
C is grams dietary fiber, and
D is grams fat.

It should be noted that the term calorie as used in physics can be defined as 4.184 absolute joules, or the amount of energy it takes to raise the temperature of one gram of water from 15° to 16° C. In the context of food, however, calories are actually equal to 1,000 standard calories (1 food calorie=1 kilocalorie). Where the term “calorie” is used herein, it is meant to define food calories, i.e., kilocalories. The compositions of the invention will preferably have Fullness Index values of at least about 4, and preferably greater than about 4.5. The most preferred formulations will have Fullness Index of about 5 and above. It is preferred that the beverages have caloric densities of from about 4 to about 10, preferably from about 6 to about 9, calories per fluid ounce. They will also preferably contain between about 10 and about 16 total grams of protein and fiber per 100 calories. The high Fullness Index values provided by the invention are evidence of both very high volume to calorie and high ratio of the total weight of fiber plus protein to calories. With these values, it is possible, indeed desirable, to consume a serving of considerable volume to provide the added benefit of volume to satiety—stomach distension—without providing calories in excess or of the wrong type, e.g., fat which can cause a craving for more fat or high glycemic index carbohydrates.

The beverages of the invention can be delicious snacks with a smooth texture and creamy mouthfeel. They can provide a smoothie-like drinking experience, with both dairy and fruit characters in some flavors. Other flavors can provide rich chocolate, coffee, tea, cola or other such flavor, which are not usually acid in taste. It is an advantage of the invention that a wide variety of delicious beverages will be possible from the core formulations which include a highly-effective combination of macro- and micronutrients that can induce satiety in human subjects. The beverages provide low energy density, but with significant protein and fiber in combination with high volume to provide satiation with minimal calorie-intake. The product has been formulated with active ingredients for satiety including fiber and protein, especially milk protein, and will beneficially employ calcium and antioxidants, especially extracted or purified epigallocatechin gallate or other catechin from green tea.

The beverages of the invention are delicious to encourage their consumption, which will provide a snack or meal introduction having a high volume to calorie ratio as well as a high ratio of (fiber plus protein) to calories. Desirably, the beverages will contain between about 10 and about 16 total grams of protein and fiber per 100 calories Each serving will preferably contain more than about 4 grams and up to about 10 grams of protein, e.g., about 5 to about 8 and most preferably about 6 grams protein, more than about 4 grams up to about 10 grams of fiber, e.g., about 5 to about 8 and most preferably about 6 grams fiber, at least about 10% of the DV (“daily value”) of calcium, e.g., at least about 20% (200 mg) of the DV for calcium, and provide less than about 100 calories, e.g., about 80 to 90 calories, most preferably with substantially no added sugars and less than about 5 grams of fat, e.g., about 3 grams of fat. In some preferred forms, the beverages will be essentially fat free, containing no added fat and a fat content arising from the incidental inclusion of fat in flavorants, etc, of less than about 2%. To help explain these amounts the description will use the term “daily value” (DV). As used throughout this description, the term “daily value” is given the meaning supplied by the U.S. FDA in a circular entitled “Daily Values Encourage Healthy Diet” by Paula Kurtzweil (http://www.fda.gov/fdac/special/foodlabel/dvs.html). The circular explains that DRVs (daily recommended values) serve as the basis for calculating percent DV. DRVs are stated to be for nutrients for which no set of standards previously existed. DRVs for the energy-producing nutrients (fat, carbohydrate, protein, and fiber) are based on the number of calories consumed per day. For labeling purposes, 2,000 calories has been established as the reference for calculating percent DVs.

Another advantageous feature of the invention is that by providing the beverage in an extremely good tasting form, the consumer is encouraged to drink a relatively large portion of the beverage having a low caloric density. The beverages have caloric densities of from about 4 to about 10, preferably from about 6 to about 9, calories per fluid ounce. The beverages are smooth textured and preferably provide from about 80 to about 100 calories for a serving size of at least about 4 fluid ounces, e.g., from about 8 to about 12 fluid ounces, and more narrowly about 10 to about 12 fluid ounces, per serving. In some cases it may be useful to provide up to about 150 calories per serving, but it is most desirable stay at or below about 100 calories.

The physical properties of the beverages are important to provide the desired high palatability to assure their consumption in the amounts necessary to provide satiation. The pH will be selected as necessary for flavor and stability and may range in acidity of from very acid, e.g., above about 3, to neutral, say above 3.5 or from about pH 3.8 to about pH 7.0. At acidic pH, the protein source is suitably stabilized, such as by food-grade gums. The pH can be achieved through the use of fruit puree alone in fruity beverages or with the addition of suitable acids and buffering salts. The preferred products will have densities of from about 8.5 to about 9 pounds per gallon (measured at about 70° F.), with the those above about 8.6 being most preferred.

It is an advantage of the invention that the beverage products can be made with superior shelf stability in terms of flavor and/or texture, and preferably can be stored at their intended conditions (which can be room temperature, e.g., 70° F., or chilled, e.g., 40° F.) for at least about 3 months and more preferably more than about 6 months. The beverages are preferably homogenized and exposed to high temperature short time heating parameters. At close to neutral pH, the products can be homogenized and then retorted or aseptically filled. Due to the choice of the ingredients and the processing by homogenization, the smooth-textured beverages will have very smooth textures. A viscosity/texture measurement is made by a TA Instruments AR2000 with concentric cylinder geometry at 4° C., a good temperature for consumption. The evaluation uses a shear rate ramp from about 0.1 to about 100 seconds⁻¹ and a power law model is fitted to the results. With a consistency coefficient of about 1000 cp and a rate index of 0.48 (no units), the perceived texture (viscosity) is preferably from about 50 to about 200 cp, e.g., about 100. Higher viscosities will give a thicker mouthfeel, which may be desirable to some people, but may leave the glass or serving container heavily coated. Alternatively, the viscosity can be within the range of from about 20 to about 50, preferably at least 25, Zahn seconds at about 42° F., when measured as follows. Place Zahn Viscometer into 42° F. product. The Zahn cup orifice is 1.5 inches. Once Zahn cup is full, lift cup out of product and start timing (in seconds). Stop timing when the Zahn cup is empty. For these products the range will typically be approximately 25-28 seconds at 42° F.

The beverages are described above and in the examples in terms of their preferred levels for each of the ingredients of the beverage. These preferred levels can be used as guidance to the skilled worker seeking to obtain variations of the formulations while maintaining the major benefits of providing an excellent beverage which has significant satiety promoting benefits. In all cases, variations from the preferred materials and procedures should be made with the objectives of the invention in mind and that the final beverage be good tasting to provide a satisfying experience coupled with a significant effect of satiety.

In the preferred beverages, the fiber, protein and calcium will be present in amounts sufficient for the beneficial effects of satiety as well as nutritional benefits over and above this objective.

It is another advantage of the invention that the beverages are designed to provide a significant source of dietary fiber. For people used to taking a fiber supplement, the invention provides a very pleasant alternative and has added advantages. Again, this advantage helps make a weight control routine self regulating. The fiber contributes to the healthfulness of the beverage as well as providing a satiety factor and adding to the enjoyable product texture and mouthfeel. Desirably, the fiber will provide up to about 50% of the daily value, and preferably from about 10 to about 30%, e.g., about 20 to about 25% of the DV, where according to the above FDA circular, the DRV for fiber is based on 11.5 g of fiber per 1,000 calories in the diet (or 23 grams for the standard 2,000 calorie diet). Preferably, the beverages will contain more than about 4 grams up to about 10 grams of fiber, e.g., 5 to 8 and most preferably about 6 grams fiber per serving.

The fiber (i.e., dietary fiber) content of the beverages of the invention can be provided from any suitable source, including that derived from various vegetable and fruit sources, including purified vegetable fiber, such as FIBRULINE™ inulin product, extracted from the roots of the chicory plant, other forms of inulin, oat fiber in various forms, various fruit pulps and vegetable pulps available largely as byproducts of juicing or other food preparation operations, such as apple pulp, citrus pulp, mango pulp, grape pulp, cranberry pulp and beet pulp; and fruit and vegetable purees, such as those prepared from fresh and/or frozen fruits and vegetables. The term fiber is given that meaning as is used in the regulations governing food labeling, namely as traditionally defined as polysaccharides and remnants of plant cell walls that are resistant to human alimentary enzymes. Dietary fiber is quantified by AOAC official methods 985.29 and 991.43. The use of purees to supply at least part of the total fiber is preferred because it supplies acid for tartness, fresh flavor, sweetness and other beneficial aspects. The most desirable products will preferably contain at least about 5%, and preferably from about 10 to about 35% of fruit puree. It is an advantage of the invention that the ability to provide real fruit in a canned beverage snack increases the appeal of consumption to the consumer and thus increases the likelihood that the weight control regimen will be successful.

Because of the near universal appeal of dairy products, and the beneficial qualities of milk proteins, the protein of choice for the beverages of the invention is milk protein. The invention can suitably employ any of the various milk protein products commercially available, but those based on yogurt and purified whey protein are preferred. Among the whey protein products are: whey protein concentrates containing from about 25 to about 80% whey protein and whey protein isolates containing at least about 90% protein. Also effective is casein in its various forms, including both lactic and acid casein as they are or resolubilized by the addition of alkali or alkali salt to form caseinates, including sodium caseinates, calcium caseinates and special blends. Other protein sources, such as pea or soy protein as isolates, concentrates, and the like, and other milk products, preferably having the fat and most of the lactose removed are also useful, as are non fat dried milk (e.g., skim, also called NFDM) and fresh milk (whether skim, whole or low fat), both whole and reduced fat, in limited amounts and depending on the characteristics needed. Another preferred form of milk protein comes in the form of yogurt, either dry or fresh and either whole or reduced fat.

The protein is desirably used in an amount sufficient to supply at least about 5%, and preferably from about 10 to about 40% of the DV for protein, e.g., about 12 to about 15%. Each serving will preferably contain more than about 4 grams and up to about 10 grams of protein, e.g., about 5 to about 8 grams, and most preferably about 6 grams, protein. And, the combined amount of protein and fiber will preferably be at least about 10 grams, and more preferably be up to about 16 grams, per serving. It is an advantage of the invention that by providing beverages meeting this criterion, in a beverage with high volume per serving and a caloric density of from about 4 to about 10 calories per fluid ounce, significant satiety benefits helpful in weight control can be achieved.

Another significant advantage of the invention, and a reason why it increases the chances that a weight conscious consumer will make it part of a weight control regimen, is the provision of a significant amount of calcium. Thus, those consumers used to taking a calcium supplement as part of their daily routine, will find it easy to supplant that with the highly beneficial composition of the invention which not only supplies the calcium, but tastes extremely good and provides dietary modulation in a very natural way. The preferred formulations will provide at least about 5% of the DV for calcium, and preferably from about 10 to about 40% of the DV for calcium, e.g., about 20 to about 30%. Any of the typical sources of dietary calcium can be employed, but it is preferred to use those from dairy sources, such as calcium lactate, calcium caseinate, other forms of dairy or dairy derived calcium, and the like. In addition to these sources, the more typical calcium carbonate from shells, and the like may also be employed.

Preferred beverages have a pleasantly sweet taste without being excessively so. Thus, to the extent that the natural sweetness of the ingredients can play a role in the final product, the preference will be toward the selection of naturally sweet ingredients and high intensity sweeteners, if desired. These selections can facilitate a preferred limitation of sugars to foster the desired low caloric content for the beverage. It is to this objective that the invention will preferably contain high intensity, low calorie sweeteners and naturally sweet food ingredients, preferably in combination with a low caloric sweetener such as erythritol. The term “sweetener” is meant to include any ingredient that provides a sweetness perception to a food. Suitable intense sweeteners include sucralose, aspartame, acesulfame-K, saccharin, cyclamate, neohesperidin dihydrochalcone, thaumatin, alitame, glycyrrhizin, and the like. Also, other low-intensity, low-calorie polyol sweeteners such as sorbitol can be employed. The polyols, also known as polyhydric alcohols, have a desirable freezing point lowering effect. Among the other suitable polyhydric alcohols are glycerol and propylene glycol. In the preferred case of the sweet polyol erythritol, the beverage compositions will contain from about 2 to about 10% by weight, e.g., from about 4 to about 9% erythritol. While not necessary in most cases, the addition of low amounts of sugars such as glucose, fructose and/or sucrose, e.g., preferably less than about 6 grams per serving, and most preferably no more than about 3 grams per serving, might be useful in particular cases. Where a sugar is desired, the satiety promoting beverages can contain up to about 11 or 12 calories for a serving, e.g., of about 8 to about 12 fluid ounces.

It is an advantage of the invention in its preferred aspects that, in addition to being very good tasting so as to encourage maintenance of a regimen for weight control, the beverages can also include active ingredients which foster weight control and yet in preferred forms can be offered as a natural product. To the great advantage of the invention is the addition to the formula of an antioxidant, especially a catechin, such as purified epigallocatechin gallate (EGCG), derived from green tea in an amount sufficient to supply from about 2 to about 4 cup equivalents, per serving. The term “cup equivalent” in this context means the amount of the active ingredient that would be present in a six ounce cup of tea brewed from about 1 teaspoon of green tea steeped in water at 80° C. (180° F.) for about 2 to about 3 minutes.

As used herein, the term “green tea” refers to leaves obtained from the genus Camellia including C. sinensis and C. assaimica, or their hybrids, for instance, freshly gathered green tea leaves, fresh green tea leaves that are dried immediately after gathering, fresh green tea leaves that have been heat treated before drying to inactivate any enzymes present, unfermented tea, instant green tea, and aqueous extracts of these leaves. Green tea materials can include tea leaves, their extracts, tea plant stems and other plant materials which are related and which have not undergone partial or substantial fermentation to create oolong or black teas. Extracts from white tea, or tea from Camelia sinensis, which has been harvested before the leaves are fully open and subject to little processing and almost no fermentation can also be used. Other members of the genus Phyllanthus, Catechu gambir or Uncaria family of tea plants can also be used. Mixtures of unfermented teas can be also used in preparing green tea extracts useful in the beverages. As used herein, the term “catechins” refers generally to catechins, epicatechins, and their derivatives. These derivatives include the sugar salts, sugar esters, and other edible physiologically available derivatives. Catechins, epicatechins, and their derivatives are present in green teas. For the purposes of the present invention, the level of catechins in the green tea solids, extracts or materials can be based of the level of the principal catechins, such as epicatechin, epigallocatechin, epicatechingallate, epigallocatechin gallate, gallocatechin and gallocatechin gallate. The purified forms as are commercially available can contain at least about 50%, and preferably about 60% or more epigallocatechin gallate or other catechin.

Another advantage of the invention is that the beverages have considerable volume and few calories. Water will comprise an amount sufficient to dissolve all of the ingredients in stable form as a ready to serve beverage. Typical water (i.e., moisture) contents include moisture from any source (including the ingredients) and will be within the range of from about 50 to about 90%, e.g., at least about 65%. The water can be supplied in any suitable form. It can be tap water, either as is or filtered or otherwise treated, or as a component of another ingredient, such as a fruit puree, fruit juice, yogurt, milk, minor ingredient premix, or the like. In some product forms, such as those intended for institutional use, the beverage may be supplied as a concentrate for final constitution to the noted moisture levels before serving.

The beverage compositions of the invention can also include additional functional ingredients, such as flavors, flavor enhancers, food-approved colors, vitamins, minerals, flow agents, etc. Typical of suitable colorants are any of those suitable for achieving the desired color. Included as representative are FD&C colors, and the like. The flavor component of the beverages will preferably be a natural flavor selected from fruit flavors, botanical flavors and mixtures thereof. Fruit and other flavors can be natural or synthetically prepared flavors made to simulate flavors derived from natural sources.

The flavors useful in the beverage concentrates are sometimes available as dry ingredients, liquids or emulsions. In any of these forms, they can be dispersed into the beverage concentrate. In addition to the active flavor itself, industrially available flavors can contain, weighting agents, emulsifiers, emulsion stabilizers, antioxidants, liquid vehicles, and the like. The particular amount of the flavor component effective for imparting flavor characteristics to the beverages and beverage concentrates of the present invention will depend upon the flavor, the flavor impression and formula flavor component. For example, the flavor component can comprise at least 0.05% by weight of the beverage composition and typically from 0.1% to about 2% by weight of the beverage (on a single strength basis). The flavors can be natural or artificial, but natural flavors are preferred. Various natural flavors are available as representing a particular flavor but are formulated with other natural flavors (WONF, e.g., strawberry WONF).

In addition to fruity flavors, such as apricot, blueberry, boysenberry, cherry, grape, grapefruit, lemon, lime, mango, orange, peach, pineapple, raspberry, strawberry and the like, the beverage concentrate compositions can contain a variety of other flavors, e.g. cola, root beer, coffee, tea, botanical flavors can include those flavors derived from nuts, bark, roots and leaves, and the like. The flavor component can comprise a single flavor or blended flavors. For example, lime and lemon flavors, cola flavors with citrus flavors to form a “cola” and pineapple and orange flavors are preferred flavor blends. Additionally preferred flavor blends can be combinations of fruit and dairy flavors, e.g., peaches and cream, and strawberries and cream, as exemplified below. Representative of other compound flavors are chocolate, Dutch chocolate, and various coffee flavors, such as café latte, cappuccino, and the like. Various tea flavors can also be compounded with other compatible flavors. These type of flavors—the chocolates, coffees and teas—are suitably prepared and packaged at pH values near neutral and can even be slightly alkaline where packaging is adapted for that purpose.

The beverage base may have an acidity and sourness suitable for the flavor impression desired and will be processed in a manner consistent with the desired pH, e.g., a pH of about 3 to pH about 7, or slightly higher. At more acidic pH values, the protein source is suitably stabilized, such as by food-grade gums. The acidity can be the natural result of the fruit based ingredients or it can be supplemented and/or adjusted with food acids and buffer salts effective for the purpose. The pH is dependent upon the particular composition of the acid component, the total amount of acids used and the sourness impression desired. Typically, the pH can range from about 3 to about 7 or more. Preferred fruity carbonated beverages have a pH of from about 3.5 to about 5.5. Other flavors not requiring the same tartness, e.g., coffee or tea drinks, will have higher pH values, such as within the range of from about 4.5 to about 6.5. As noted, the chocolate, coffee and tea flavors are suitable at pH values near neutral and can even be slightly alkaline

Of the acids that can be added, citric acid is preferred. Inorganic acids, such as phosphoric, can be used with minimal impact on caloric content, but are used where their flavors are best tolerated. However, any of the suitable food acidulents can be employed, e.g., a member selected from the group consisting of adipic acid, citric acid, fumaric acid, lactic acid, malic acid (e.g., dl malic), tartaric acid, and mixtures of at least two of these. The terms “food acid” and “food acidulent” are used herein to mean carboxylic acids, such as those mentioned, which are useful for imparting a tart taste to foods. These acids are compatible with food acceptable inorganic acids such as phosphoric, hydrochloric, and the like. It is sometimes desirable to add one or more buffering salts compatible with the acid and other ingredients. Suitable buffering salts can include one or more members selected from the group consisting of the alkaline earth and/or alkali metal salts of calcium, sodium and potassium salts of the listed food acids. Among the specific useful weak acid salts are sodium citrate, potassium citrate, disodium phosphate, dipostassium phosphate, monocalcium phosphate, tricalcium phosphate, and mixtures of at least two of these.

The beverages of the invention can comprise other optional nutritional ingredients. Other supplements, including dietary supplements, e.g., stevia, can be employed as desirable. The beverages can be fortified with from about 0 to about 110%, e.g., from about 20 to 100%, of the DV of selected vitamins and minerals, provided that such vitamins and minerals do not substantially alter the desired properties of the beverage and that such vitamins and minerals are chemically and physically compatible with the other essential components of beverage. Especially preferred are vitamin A (e.g., vitamin A palmitate), provitamins thereof (e.g., beta.-carotene), vitamin B1 (e.g., thiamin HCl) and vitamin C (i.e., ascorbic acid), although it is understood that other vitamins and minerals can also be used.

The beverage products of the invention are most desirable when processed to achieve a smooth, homogeneous texture having good shelf stability. They taste best in preferred forms when chilled to refrigerated temperature. When refrigerated, the products will remain stable and suitable for consumption for at least one month, and preferably for at least 3 months and more preferably more than about 6 months. Desirably, they will also have extended shelf life when not refrigerated. The pH can be achieved through the use of fruit puree alone or suitable acids and buffering salts can be added. For shelf stability, the beverage is homogenized such as using a homogenizer such as by Niro Soavi or APV Gaulin, and exposed to high temperature short time heating parameters. At close to neutral pH, the product can be homogenized and then retorted or aseptically filled.

Again, an advantage of the invention is that it provides a delicious beverage that affords a pleasant and convenient experience in every way—to encourage maintenance of a simple regimen to enable weight control. Packaging can be in cartons as used for dairy products. Cans and bottles are also suitable. The choice of packaging will largely be a matter of choice and will preferably have a convenient opening feature to facilitate snack use without the need for special opening devices.

It is an important aspect of the invention to provide a regimen for weight control. The regimen of the invention provides a solution to weight loss and weight management with a simple self regulatory feature—it is so good tasting it will be easy to remember to take it as a treat. The regimen, from one perspective, comprises: consuming one satiety promoting beverage comprising fiber, protein, especially from milk, calcium, sweetener and preferably an antioxidant, e.g., purified epigallocatechin gallate derived from green tea, the ingredients being employed in amounts sufficient that a single serving will provide a feeling of fullness and satiety to the person consuming it, both initially and after a period of at least one hour; and, eating a regular meal within one hour of completion of the beverage. The amounts of the individual ingredients will preferably be within the ranges described above. The beverage will preferably provide the ingredients in relative and total amounts effective to provide a measurable feeling of satiety and an increase in CCK serum levels. According to the invention the noted measurable feeling of satiety can be measured by noting the patients' subjective impressions in a format to provide statistical significance or by any of the objective criteria identified in the references noted above. The satiety will be felt both initially and after a period of at least one hour, and preferably for two hours.

In addition to the above, it is preferred to utilize the above regimen on a regular basis of at least about 5 times per week. It is an advantage of the invention that a weight conscious individual utilizing beverage in a regimen will be able to make weight control more simple and more pleasant. By providing an immediate satiety response to consumption, the person can drink the beverage directly before eating and still have benefit. By providing a long lasting response, timing of consumption should not be a separate cause of stress. Preferably, the amounts of the various ingredients will be effective to result in the person consuming the beverage taking in less food in terms of weight and/or calories. And, it is also an advantage of the invention that the beverage can be consumed as a snack to curb hunger for a period of from about 1 to about 2 hours.

The following examples are provided to further illustrate and explain the invention, without being limiting in any regard. Unless otherwise indicated, all parts and percentages are based on the weight of the composition at the particular point of reference.

### EXAMPLE 1

In this example, a satiety beverage is prepared from a composition as shown in the following table.

| Ingredients | Weight | Percent |
|---|---|---|
| Water | 245.00 | 53.12% |
| Strawberry Puree, seedless | 95.00 | 20.60% |
| Yogurt, plain | 75.00 | 16.26% |
| Erythritol | 30.00 | 6.50% |
| Chicory Root Fiber, 94% dietary fiber | 5.00 | 1.08% |
| Whey Protein Concentrate, 98% protein | 4.50 | 0.98% |
| Calcium Lactate | 2.50 | 0.54% |
| Pectin | 0.67 | 0.15% |
| Dipotassium Phosphate | 0.60 | 0.13% |
| Gum Blend - xanthan/guar | 0.50 | 0.11% |
| Potassium Citrate | 0.50 | 0.11% |
| Natural Red Color | 0.38 | 0.08% |
| EGCG Extract | 0.38 | 0.08% |
| Strawberry WONF | 0.45 | 0.10% |
| Citric Acid, anhydrous | 0.36 | 0.08% |
| Gellan Gum | 0.07 | 0.02% |
| Stevia | 0.11 | 0.02% |
| Salt | 0.10 | 0.02% |
| Vanilla WONF | 0.10 | 0.02% |
| | 461.22 | 100.00% |

These ingredients are processed according to the following procedure:

1. Mix together pectin and gum blend.

2. Combine rest of dry ingredients.

3. Combine all wet ingredients except water and strawberry puree.

4. In Dispermat, add water along with pectin and gum blend.

5. Hydrate for 10 minutes in Dispermat.

6. Add rest of dry ingredients and turn on heating element.

7. Heat to 195° F.

8. Place in homogenizer at 2500 psi and 500 psi.

9. Cool.

10. Package in sealed, coated paperboard cartons.

### EXAMPLE 2

In this example, a mango beverage is prepared from the following ingredients and the process as described in Example 1.

| Ingredients | Weight | Percent |
|---|---|---|
| Water | 233.00 | 60.79% |
| Yogurt, plain | 50.00 | 13.04% |
| Mango Puree | 50.00 | 13.04% |
| Erythritol | 30.00 | 7.83% |
| Chicory Root Fiber, 94% dietary fiber | 5.00 | 1.30% |
| Whey Protein Concentrate, 98% protein | 6.00 | 1.57% |
| Calcium Lactate | 2.20 | 0.57% |
| Mango Flavor | 1.94 | 0.51% |
| Natural Yellow Color | 1.60 | 0.42% |
| Pectin | 1.00 | 0.26% |
| Citric Acid | 0.67 | 0.17% |
| Xanthan Gum | 0.50 | 0.13% |
| Postassium Citrate | 0.50 | 0.13% |
| EGCG | 0.44 | 0.11% |
| Gellan Gum | 0.15 | 0.04% |
| Stevia | 0.11 | 0.03% |
| Vanilla Flavor | 0.10 | 0.03% |
| Salt | 0.10 | 0.03% |
| | 383.31 | 100.00% |

### EXAMPLE 3

In this example, a blueberry beverage is prepared from the following ingredients and the process as described in Example 1.

| Ingredients | Weight | Percent |
|---|---|---|
| Water | 233.00 | 71.89% |
| Yogurt, plain | 25.00 | 7.71% |
| Blueberry Puree | 15.00 | 4.63% |
| Erythritol | 33.50 | 10.34% |
| Chicory Root Fiber, 94% dietary fiber | 4.00 | 1.23% |
| Whey Protein Concentrate, 98% protein | 4.50 | 1.39% |
| Calcium Lactate | 2.20 | 0.68% |
| Blueberry Flavor | 2.30 | 0.71% |
| Pectin | 1.40 | 0.43% |
| Citric Acid | 0.65 | 0.20% |
| Xanthan Gum | 0.50 | 0.15% |
| Postassium Citrate | 0.50 | 0.15% |
| EGCG | 0.44 | 0.14% |
| Gellan Gum | 0.18 | 0.06% |
| Stevia | 0.13 | 0.04% |
| Cream Flavor | 0.30 | 0.09% |
| Salt | 0.50 | 0.15% |
| | 324.10 | 99.99% |

### EXAMPLE 4

In this example, a peach beverage is prepared from the following ingredients and the process as described in Example 1.

| Ingredients | Weight | Percent |
|---|---|---|
| Water | 170.00 | 47.33% |
| Peach Puree | 100.00 | 27.84% |
| Yogurt, plain | 75.00 | 20.88% |
| Nutriose, 85% dietary fiber | 5.00 | 1.39% |
| Whey Protein Concentrate, 98% protein | 3.60 | 1.00% |
| Pectin | 2.00 | 0.56% |
| Gum Blend - xanthan/guar | 0.50 | 0.14% |
| Potassium Citrate | 0.50 | 0.14% |
| Natural Color | 0.80 | 0.22% |
| EGCG Extract | 0.35 | 0.10% |
| Peach Flavor WONF | 0.99 | 0.28% |
| Sucralose | 0.21 | 0.06% |
| Citric Acid, anhydrous granular | 0.11 | 0.03% |
| Salt | 0.10 | 0.03% |
| | 359.16 | 100.00% |

### EXAMPLE 5

In this example, a satiety beverage with a flavor described as Peaches & Cream is prepared from the composition shown in the following table.

| Ingredients | % Solids | Percent |
|---|---|---|
| Water | | 46.721% |
| Erythritol | 95.00% | 8.234% |
| Pectin | 95.00% | 0.251% |
| Xanthan/Guar Blend | 99.00% | 0.140% |
| Gellan Gum | 95.00% | 0.064% |
| Peach Puree | 10.00% | 27.429% |
| Whey Protein Isolate | 95.00% | 0.950% |
| EGCG | 95.00% | 0.080% |
| Stevia | 95.00% | 0.027% |
| Fibruline | 99.00% | 1.003% |
| Calcium Lactate | 99.00% | 0.271% |
| Dipotassium Phosphate | 99.00% | 0.140% |
| Potassium Citrate | 99.00% | 0.140% |
| Salt | 99.00% | 0.030% |
| Yogurt, plain, Non-Kosher | 8.75% | 13.720% |
| Shady Tangerine Color | 99.00% | 0.140% |
| Peach Flavor WONF | 10.00% | 0.470% |
| Vanilla Flavor | 10.00% | 0.100% |
| Cream Flavor | 10.00% | 0.080% |
| Antifoam | 95.00% | 0.010% |
| | | 100.000% |

These ingredients are processed according to the following procedure:
1. Preblend Gums with portion of Erythritol
2. Preblend Whey with portion of Erythritol
3. Preblend EGCG with portion of Erythritol
4. Add Cold Water to blend tank with a Scott Turbon Mixer. Hold 2 gal (16.6 lbs) for rinsing.
5. With agitation on, add Gum Blend to water and mix until no lumps remain. Note: Adjust agitation to minimize air incorporation
6. Add Peach Puree.
7. Add Whey Blend, EGCG Blend and remaining dry ingredients. Add Antifoam.
8. Add Yogurt.
9. Add Peach, Vanilla and Cream Flavors and Color.
10. Blend for 10 min.
11. Sample for Brix, pH and Solids analysis.
12. Process through Tetra Pak Steam Injection Unit at 289° F. for 2.12 seconds trim cool to 170° F., homogenize at 2500 psi total/500 psi 2nd stage
13. Cool to 90° F. and Fill into 250 ml Tetra Prisma (TPA/19) with high barrier film K-Film
14. Refrigerate

The beverage made as above is packaged in 11 ounce (340 gram) coated paperboard containers. Each container was a recommended serving and contained about 6 grams protein, 6 grams fiber, 20% of the DV for calcium and provided 90 calories with no added sugars and less than 3 grams of fat. In addition, the beverage as batched and prior to packaging exhibited a pH of 4.80 and after packaging, the following properties:

| Property | Value |
|---|---|
| Fullness Index | 5.5 |
| Density (pounds per gallon) | 8.648 |
| Viscosity, Zahn @43° F. (sec) | 26 |
| pH | 4.35-4.38 |

### EXAMPLE 6

In this example, a satiety beverage with a flavor described as Strawberries & Cream is prepared from the composition shown in the following table.

| Ingredients | % Solids | Percent |
|---|---|---|
| Water | | 58.325% |
| Erythritol | 95.00% | 8.423% |
| Pectin | 95.00% | 0.138% |
| Xanthan/Guar Blend | 99.00% | 0.129% |
| Gellan Gum | 95.00% | 0.066% |
| Whey Protein Isolate | 95.00% | 1.372% |
| EGCG | 95.00% | 0.076% |
| Stevia | 95.00% | 0.031% |
| Fibruline | 99.00% | 1.012% |
| Calcium Lactate | 99.00% | 0.302% |
| Dipotassium Phosphate | 99.00% | 0.147% |
| Potassium Citrate | 99.00% | 0.128% |
| Salt | 99.00% | 0.147% |
| Strawberry Puree, Seedless | 8.00% | 22.873% |
| Yogurt, plain, Non-Kosher | 8.75% | 6.292% |
| Vanilla Flavor WONF | 10.00% | 0.030% |
| Strawberry Flavor | 10.00% | 0.430% |
| Valencia Red | 99.00% | 0.069% |
| Antifoam | 95.00% | 0.010% |
| | | 100.000% |

These ingredients are processed according to the following procedure:
1. Preblend Gums with portion of Erythritol
2. Preblend Whey with portion of Erythritol
3. Preblend EGCG with portion of Erythritol
4. Add portion of the Cold Water to Milk Can with an Industrial Immersion Blender.
5. With agitation on, add Gum Blend to water in Milk Can and mix until no lumps remain.
6. Along with remaining water, transfer to Batch Tank with Scott Turbon Mixer. Hold 2 gal (16.6 lbs) for rinsing.
7. Add Whey Blend, EGCG Blend and remaining dry ingredients. Add Antifoam.
8. Add Strawberry Puree
9. Add Yogurt.
10. Add Strawberry and Vanilla Flavors.
11. Blend for 10 min.
12. Sample for Brix, pH and Solids analysis.
13. Process through Tetra Pak Steam Injection Unit at 289° F. for 2.12 seconds trim cool to 170° F., homogenize at 2500 psi total/500 psi 2nd stage
14. Cool to 110° F. and Fill into 250 ml Tetra Prisma (TPA/19) with Wide type Film
15. Refrigerate

The beverage made as above is packaged in 11 ounce (340 gram) coated paperboard containers. Each container was a recommended serving and contained about 6 grams protein, 6 grams fiber, 20% of the DV for calcium and provided 80 calories with no added sugars and less than 3 grams of fat. In addition, the beverage as batched and prior to packaging exhibited a pH of 4.75 and after packaging, the following properties:

| Property | Value |
|---|---|
| Fullness Index | 6.2 |
| Density (pounds per gallon) | 8.730 |
| Viscosity, Zahn @43° F. (sec) | 30-32 |
| pH | 4.47 |

### EXAMPLE 7

In this example, a satiety beverage with a flavor described as Caffe Latte is prepared from the composition shown in the following table.

| Ingredients | % Solids | Percent |
|---|---|---|
| Water | | 74.969% |
| Erythritol | 95.00% | 8.457% |
| Pectin | 95.00% | 0.241% |
| Xanthan/Guar Blend | 99.00% | 0.133% |
| Gellan Gum | 95.00% | 0.210% |
| Whey Protein Isolate | 95.00% | 1.150% |
| EGCG Extract | 95.00% | 0.075% |
| Stevia | 95.00% | 0.028% |
| Fibruline | 99.00% | 1.575% |
| NFDM | 99.00% | 0.578% |
| Maltodextrin | 99.00% | 1.400% |
| Dark Roast Decaf coffee | 95.00% | 0.500% |
| Cream powder | 10.00% | 0.555% |
| Calcium Lactate | 99.00% | 0.275% |
| Dipotassium Phosphate | 99.00% | 0.145% |
| Potassium Citrate | 99.00% | 0.120% |
| Salt | 99.00% | 0.072% |
| Decaf Coffee Conc | 13.00% | 1.975% |
| Yogurt, plain, Non-Kosher | 8.75% | 7.250% |
| Vanilla Flavor WONF | 10.00% | 0.193% |
| Cream Flavor | 10.00% | 0.089% |
| Antifoam | 95.00% | 0.010% |
| | | 100.000% |

These ingredients are processed according to the following procedure:
1. Preblend Gums with portion of Erythritol
2. Preblend Whey with portion of Erythritol
3. Preblend EGCG with portion of Erythritol
4. Add portion of the Cold Water to Milk Can with an Industrial Immersion Blender.
5. With agitation on, add Gum Blend to water in Milk Can and mix until no lumps remain.
6. Along with remaining water, transfer to Batch Tank with Scott Turbon Mixer. Hold 2 gal (16.6 lbs) for rinsing.
7. Add Whey Blend, EGCG Blend and remaining dry ingredients. Add Antifoam.
8. Add Yogurt and Coffee Concentrate.
9. Add Vanilla and Cream Flavors.
10. Blend for 10 min.
11. Sample for Brix, pH and Solids analysis.
12. Process through Tetra Pak Steam Injection Unit at 289° F. for 2.12 seconds trim cool to 170° F., homogenize at 2500 psi total/500 psi 2nd stage
13. Cool to 110° F. and Fill into 250 ml Tetra Prisma (TPA/19) with Wide type Film
14. Refrigerate

The beverage made as above is packaged in 11 ounce (340 gram) coated paperboard containers. Each container was a recommended serving and contained about 6 grams protein, 6 grams fiber, 20% of the DV for calcium and provided 90 calories with no added sugars and less than 3 grams of fat. In addition, the beverage as batched and prior to packaging exhibited a pH of 5.65 and after packaging, the following properties:

| Property | Value |
|---|---|
| Fullness Index | 5.5 |
| Density (pounds per gallon) | 8.730 |
| Viscosity, Zahn @42° F. (sec) | 28-30 |
| pH | 6.80 |

### EXAMPLE 8

In this example, a satiety beverage with a flavor described as Dutch Chocolate is prepared from the composition shown in the following table.

| Ingredients | % Solids | Percent |
|---|---|---|
| Water | | 75.472% |
| Erythritol | 95.00% | 9.056% |
| Pectin | 95.00% | 0.252% |
| Xanthan/Guar Blend | 99.00% | 0.119% |
| Gellan Gum | 95.00% | 0.211% |
| Whey Protein Isolate | 95.00% | 0.950% |
| EGCG Extract | 95.00% | 0.077% |
| Cocoa Powder | 95.00% | 0.956% |
| Cocoa Powder, alkalized | 95.00% | 1.019% |
| Stevia | 95.00% | 0.030% |
| Fibruline | 99.00% | 0.956% |
| NFDM | 99.00% | 0.516% |
| Maltodextrin | 99.00% | 1.736% |
| Calcium Lactate | 99.00% | 0.252% |
| Dipotassium Phosphate | 99.00% | 0.151% |
| Potassium Citrate | 99.00% | 0.126% |
| Salt | 99.00% | 0.075% |
| Yogurt, plain, Non-Kosher | 8.75% | 7.295% |
| Nat Dark Chocolate Flavor | 10.00% | 0.528% |
| Vanilla WONF | 10.00% | 0.113% |
| Cream Flavor | 10.00% | 0.101% |
| Antifoam | 95.00% | 0.010% |
| | | 100.000% |

These ingredients are processed according to the following procedure:
1. Preblend Gums with portion of Erythritol
2. Preblend Whey with portion of Erythritol
3. Preblend EGCG with portion of Erythritol
4. Pre-hydrate Cocoa Powders in 40 lbs Hot Water in Milk can with blender.
5. Add 57.4 gals (477.6 lbs) Cold Water to blend tank with a Scott Turbon Mixer. Hold 2 gal (16.6 lbs) for rinsing.
6. With agitation on, add Gum Blend to water and mix until no lumps remain. Note: Adjust agitation to minimize air incorporation
7. Add Whey Blend, EGCG Blend and remaining dry ingredients. Add Antifoam.
8. Add Pre-hydrated Cocoa mixture.
9. Add Yogurt.
10. Add Chocolate, Vanilla and Cream Flavors.
11. Blend for 10 min.
12. Sample for Brix, pH and Solids analysis.
13. Process through Tetra Pak Steam Injection Unit at 293° F. for 2.12 seconds trim cool to 170° F., homogenize at 2500 psi total/500 psi 2nd stage
14. Cool to 90° F. and Fill into 250 ml Tetra Prisma (TPA/19) with high barrier film K-Film
15. Refrigerate

The beverage made as above is packaged in 11 ounce (340 gram) coated paperboard containers. Each container was a recommended serving and contained about 6 grams protein, 6 grams fiber, 20% of the DV for calcium and provided 90 calories with no added sugars and less than 3 grams of fat. In addition, the beverage as batched and prior to packaging exhibited a pH of 6.17 and after packaging, the following properties:

| Property | Value |
|---|---|
| Fullness Index | 5.5 |
| Density (pounds per gallon) | 8.850 |
| Viscosity, Zahn @42° F. (sec) | 30-32 |
| pH | 6.34-6.37 |

The invention fills a need for a food choice that is low in calories while providing high nutritional value and a feeling of fullness. In one aspect, the invention provides a method of controlling calories between meals without feeling hungry. When used in the described regimen it can greatly moderate hunger cravings between meals and, therefore, eliminate overeating at subsequent meals. When used in a somewhat less rigorous fashion as a between meal snack, it can also have positive benefits of providing satisfaction without the calories attendant other snack choices. Also, many weight conscious individuals will find that it is desirable to maintain daily records of the consumption of the beverage and other important related factors, including exercise and servings of fruit and vegetables on the positive side and desserts and high calorie snacks on the negative side.

The above description is for the purpose of teaching the person of ordinary skill in the art how to practice the invention. It is not intended to detail all of those obvious modifications and variations, which will become apparent to the skilled worker upon reading the description. It is intended, however, that all such obvious modifications and variations be included within the scope of the invention which is defined by the following claims. The claims are meant to cover the claimed components and steps in any sequence which is effective to meet the objectives there intended, unless the context specifically indicates the contrary.

1. A satiety promoting beverage, comprising: fiber, protein, calcium and sweetener, wherein each serving of from about 4 to about 12 fluid ounces contains more than about 4 grams of protein, more than about 4 grams of fiber, with a combined amount of protein and fiber of from about 10 to about 16 grams, and provides less than about 100 calories and has a caloric density of from about 4 to about 10 calories per fluid ounce.

2. A satiety promoting beverage according to claim 1, wherein the beverage provides about 5 to about 8 grams protein, about 5 to about 8 grams fiber and at least about 10% of the DV of calcium, in a serving of about 8 to about 12 ounces of beverage.

3. A satiety promoting beverage according to claim 1, wherein the beverage provides about 6 grams of fiber, about 6 grams of protein and from about 80 to about 100 calories per about 10 to about 12 ounces of beverage.

4. A satiety promoting beverage according to claim 3, wherein the beverage has a caloric density of from about 6 to about 9 calories per fluid ounce.

5. A satiety promoting beverage according to claim 1, wherein the beverage has a Fullness Index of at least about 4, where the Fullness Index=(41.7/(Aˆ0.7))+(0.05*B)+(6.17*(EXP(−4))*(Cˆ3)−(7.25*(EXP(−6))*D)+0.617)
where for each 100 g of beverage,
A is calories
B is grams protein
C is grams dietary fiber, and
D is grams fat.

6. A satiety promoting beverage according to claim 5, wherein the Fullness Index is greater than about 4.5.

7. A satiety promoting beverage according to claim 1, wherein the beverage is fruit flavored, contains fruit puree and has a pH in the range of from about 3.5 to about 5.5.

8. A satiety promoting beverage according to claim 1, wherein the beverage is chocolate flavored and has a pH in the range of from about 4.5 to about 6.5.

9. A satiety promoting beverage according to claim 1, which further includes a catechin in an amount sufficient to supply from about 2 to about 4 cup equivalents per serving.

10. A satiety promoting beverage according to claim 9, wherein the catechin comprises one or more of epicatechin, epigallocatechin, epicatechingallate, epigallocatechin gallate, gallocatechin and gallocatechin gallate.

11. A satiety promoting beverage according to claim 1, which exhibits a viscosity within the range of from about 20 to about 50 Zahn seconds at about 42° F.

12. A satiety promoting beverage according to claim 1, which is shelf stable for at least about six months at about 70° F.

13. A satiety promoting beverage, comprising: fiber, protein and sweetener, the ingredients being employed in amounts sufficient to provide a Fullness Index of at least about 4, where the Fullness Index=(41.7/(Aˆ0.7))+(0.05*B)+(6.17*(EXP(−4))*(Cˆ3)−(7.25*(EXP(−6))*D)+0.617)
where for each 100 g of beverage,
A is calories
B is grams protein
C is grams dietary fiber, and
D is grams fat.

14. A satiety promoting beverage according to claim 13, wherein each serving of from about 4 to about 12 fluid ounces contains more than about 4 grams of protein, more than about 4 grams of fiber, and at least about 10% of the DV of calcium, while providing less than about 150 calories.

15. A satiety promoting beverage according to claim 13, wherein the beverage has a caloric density of from about 4 to about 10 calories per fluid ounce.

16. A satiety promoting beverage according to claim 13, wherein the beverage has a caloric density of from about 6 to about 9 calories per fluid ounce.

17. A satiety promoting beverage according to claim 16, wherein the beverage contains between about 10 and about 16 total grams of protein and fiber per 100 calories.

18. A satiety promoting beverage according to claim 17, wherein the beverage has a Fullness Factor of at least about 4.5.

19. A satiety promoting beverage according to claim 13, wherein the amounts of the various ingredients will be effective to result in the person consuming the beverage taking in less food in terms of weight and/or calories at a meal taken within about two hours of consumption of the beverage.

20. A satiety promoting beverage according to claim 13, wherein the amounts of the ingredients are effective to provide a measurable feeling of satiety and an increase in CCK serum levels.

21. A satiety promoting beverage according to claim 13, wherein: the fiber is present in an amount sufficient to supply from about 4 to about 10 grams fiber, milk protein is present in an amount sufficient to supply from about 4 to about 10 grams protein per serving, and the ingredients have been homogenized to provide a smooth textured beverage with a creamy mouthfeel and from about 80 to about 100 calories for a serving of about 8 to about 12 fluid ounces.

22. A satiety promoting beverage according to claim 21, wherein the Fullness Index is about 5.

23. A satiety promoting beverage according to claim 22, wherein the amounts of the ingredients are effective to provide a feeling of satiety and an increase in CCK serum levels.

24. A regimen for weight control, which comprises: prior to a meal, consuming one satiety promoting beverage comprising fiber, protein, calcium and sweetener, the ingredients, wherein each serving of from about 4 to about 12 fluid ounces contains more than about 4 grams of protein and more than about 4 grams of fiber, with a combined amount of protein and fiber of from about 10 to about 16 grams, and provides less than about 100 calories and has a caloric density of from about 4 to about 10 calories per fluid ounce.

25. A regimen according to claim 24, wherein the above regimen is repeated on a regular basis of at least about 5 times per week.

26. A regimen according to claim 24, wherein the beverage is consumed up to about 2 hours prior to eating a meal.

27. A regimen according to claim 24, wherein the beverage has a Fullness Index of at least about 4, where the Fullness Index=(41.7/(Aˆ0.7))+(0.05*B)+(6.17*(EXP(−4))*(Cˆ3)−(7.25*(EXP(−6))*D)+0.617)
where for each 100 g of beverage,
A is calories
B is grams protein
C is grams dietary fiber, and
D is grams fat.

28. A regimen according to claim 27, wherein the Fullness Index is about 5.

29. A satiety promoting beverage, comprising: fiber, protein, calcium and sweetener, the ingredients being employed in amounts sufficient that a single serving will provide a feeling of fullness and satiety to the person consuming it, both initially and after a period of at least one hour.

30. A satiety promoting beverage, comprising:
fiber in an amount sufficient to supply from 10 to 30% of the DV for fiber;
milk protein in an amount sufficient to supply from 4 to 10 grams protein;
calcium in an amount sufficient to supply from 10 to 40% of the DV for calcium;
a low calorie sweetener and,
purified epigallocatechin gallate derived from green tea in an amount sufficient to supply from 2 to 4 cup equivalents,
wherein the ingredients have been homogenized and packaged to provide a smooth textured beverage with a creamy mouthfeel and from about 50 to about 150 calories per serving of from about 4 to 12 ounces.

31. A satiety promoting beverage according to claim 30, wherein: the beverage contains up to about 12 calories for a serving of about 8 to about 12 fluid ounces.
